(19) United States
(12) Patent Application Publication
(10) Pub. No.: US 2013/0014750 A1
(43) Pub. Date: Jan. 17, 2013

(54) Intubation Apparatus

(71) Applicant: Soheil Etesham, Bakersfield, CA (US)

(72) Inventor: Soheil Etesham, Bakersfield, CA (US)

(21) Appl. No.: 13/182,843

(22) Filed: Jul. 14, 2011

(51) Int. Cl. A61M 16/00

(52) U.S. Cl. 128/200.26

(57) ABSTRACT

An intubation apparatus comprising an elongate support member, a blade element, a guiding stylet, and a tubular sheath is provided. The elongate support member comprises a proximal section, and a distal section curvedly extending from the proximal section. The blade element is configured on the curvedly extended distal section of the elongate support member for maneuvering the intubation apparatus into a patient's oral cavity. A guiding stylet is detachably attached to the elongate support member and substantially follows curvature of the elongate support member and the blade element. The tubular sheath, slidably and coaxially enclosing the guiding stylet, extends along a length of the guiding stylet and advances an extended distance into the trachea by sliding over the length of the guiding stylet and then over an adjustable distal tip of the guiding stylet. An endotracheal tube is slidably and coaxially guided over the advanced tubular sheath into the trachea.

### BACKGROUND

Medical procedures, for example, surgery, treatment of certain pulmonary diseases, etc., require the introduction of endotracheal tubes into the trachea of a patient to maintain an open airway, or to serve as a conduit for administering certain drugs. Frequently, the endotracheal tubes are used in operating rooms during surgery and in locations outside of operating rooms, for example, surgical suites such as emergency rooms, other remote locations, etc. Limited or poor visibility in an area inside the patient's oral cavity where the endotracheal tubes are inserted makes it difficult to accurately position the endotracheal tubes. Furthermore, positioning and maneuvering endotracheal tubes in patients having small mouth openings is a difficult task. Improper positioning of the endotracheal tubes or inability to secure an airway can lead to adverse consequences for the patient. Hence, there is a need for providing unobstructed visibility for the introduction of the endotracheal tubes into vocal cords of a patient for performing intubation with minimal effort.

During particularly difficult intubations, once the vocal cords are visualized, it is imperative to intubate as expeditiously as possible, which necessitates pushing a stylet forward and deep into the vocal cords. Conventional endotracheal tubes that provide stylets are often not effective in an intubation procedure as they do not provide additional extendibility. Hence, there is a need for an apparatus that allows a medical practitioner to extend and protrude a stylet past the vocal cords, and into the trachea. Moreover, it is difficult to control the movement of the stylet, as the stylet may move in any direction in the oral cavity and away from a target area. Therefore, there is also a need for controlling and directing the stylet to the target area in a patient's trachea for performing controlled intubation.

Moreover, conventional endotracheal tubes require a physician to lean forward and flex the arm holding the stylet to make a fast sweep forward and complete the intubation. These maneuvers consume valuable time, and under certain circumstances the physician can lose visualization of the vocal cords and can jeopardize the patient's life. Hence, there is a need for a stylet that is controllably extendable at the tip to enable a physician to complete the intubation without having to constantly move either the stylet or a handle of the endotracheal tube in order to obtain a better view of the intubation area.

Hence, there is a long felt but unresolved need for an intubation apparatus that provides unobstructed visibility of a patient's vocal cords, and a guiding stylet that can be maneuvered up and down a patient's oral cavity and is also extendable for additional maneuverability. Furthermore, there is a need for an intubation apparatus that enables a medical practitioner to perform controlled intubation at a target area in a patient's trachea with minimal movement and with greater precision.

### SUMMARY OF THE INVENTION

This summary is provided to introduce a selection of concepts in a simplified form that are further described in the detailed description of the invention. This summary is not intended to identify key or essential inventive concepts of the claimed subject matter, nor is it intended for determining the scope of the claimed subject matter.

The apparatus and method disclosed herein address the above stated need for an intubation apparatus that provides unobstructed visibility of a patient's vocal cords and a guiding stylet that can be maneuvered up and down a patient's oral cavity and is also extendable for additional maneuverability. The intubation apparatus disclosed herein enables a medical practitioner to perform controlled intubation at a target area in a patient's trachea with minimal movement and with greater precision. The intubation apparatus disclosed herein is useful for performing controlled intubation in patients having small mouth openings.

The intubation apparatus disclosed herein comprises an elongate support member, a blade element, a guiding stylet, and a tubular sheath. The elongate support member comprises a proximal section and a distal section. The distal section of the elongate support member curvedly extends from the proximal section of the elongate support member.

The blade element is configured on the curvedly extended distal section of the elongate support member for maneuvering the intubation apparatus into an oral cavity of a patient. The blade element has a curvature of, for example, about 20 degrees to about 40 degrees. In an embodiment, the intubation apparatus disclosed herein further comprises a viewing lens housed on a distal end of the blade element for viewing the vocal cords and the trachea of the patient. The viewing lens is, for example, a fiber optic lens, an infrared imaging lens, an ultrasound imaging lens, etc. The infrared imaging lens or the ultrasound imaging lens provides an unobstructed view of the patient's trachea, as these lenses do not distort or block the view in the presence of secretions, blood or severe inflammatory reactions. The distal end of the blade element is curved slightly upwards and houses the viewing lens.

In an embodiment, the intubation apparatus disclosed herein further comprises one or more channels defined within the elongate support member for providing a passageway from the proximal section of the elongate support member to the distal section of the elongate support member. For example, one of the channels is a light channel defined within the elongate support member for transmitting light from a light source positioned on the proximal section of the elongate support member to a light port housed on the distal end of the blade element configured on the distal section of the elongate support member. In another example, one of the channels is a viewing lens channel defined within the elongate support member for guiding light from the viewing lens housed on the distal end of the blade element to a viewing scope or a monitor operably coupled to the proximal section of the elongate support member.

The guiding stylet is detachably attached to the elongate support member. The guiding stylet substantially follows the curvature of the elongate support member and the blade element for guiding an endotracheal tube into the patient's trachea. The guiding stylet comprises an adjustable distal tip at a distal end of the guiding stylet. The tubular sheath slidably and coaxially encloses the guiding stylet. The tubular sheath extends along a length of the guiding stylet and advances an extended distance into the trachea by sliding over the length of the guiding stylet and then over the adjustable distal tip of the guiding stylet. For example, the tubular sheath can be extended and protruded several centimeters in the patient's trachea. The tubular sheath extends beyond the blade element and advances the extended distance into the trachea. The endotracheal tube is slidably and coaxially guided over the advanced tubular sheath into the trachea.

The intubation apparatus disclosed herein further comprises a control lever extending outwardly from a proximal end of the guiding stylet for controlling movement and orientation of the adjustable distal tip of the guiding stylet. The adjustable distal tip of the guiding stylet bends from, for example, about 120 degrees in an upward direction to about −30 degrees in a downward direction. Bending of the guiding stylet provides easier access of the guiding stylet into the vocal cords, while sliding the tubular sheath over the length of the guiding stylet and then over the adjustable distal tip of the guiding stylet enables a medical practitioner to complete intubation without requiring the medical practitioner to constantly move either the guiding stylet or the elongate support member of the intubation apparatus to obtain a better view of the patient's vocal cords. The advancement of the tubular sheath over the adjustable distal tip of the guiding stylet to the extended distance controllably directs the guiding stylet to the target area in the patient's trachea for performing controlled intubation.

In an embodiment, the intubation apparatus disclosed herein further comprises a receiving member defined on an outer surface of the elongate support member for receiving the guiding stylet. The receiving member further receives the tubular sheath and the endotracheal tube that slidably and coaxially enclose the guiding stylet. In another embodiment, the guiding stylet slidably and coaxially enclosed by the tubular sheath is contained within one of multiple channels defined within the elongate support member. The intubation apparatus disclosed herein further comprises one or more openings defined on an outer surface of the elongate support member for inserting the guiding stylet and the tubular sheath that slides over the length of the guiding stylet.

In an embodiment, the intubation apparatus disclosed herein further comprises a viewing scope operably coupled to the proximal section of the elongate support member for enabling viewing of the patient's vocal cords and trachea via the viewing lens. In another embodiment, the intubation apparatus disclosed herein further comprises a monitor operably coupled to the proximal section of the elongate support member for enabling viewing of the patient's vocal cords and trachea via the viewing lens. The intubation apparatus disclosed herein further comprises a light source positioned on the proximal section of the elongate support member and below the viewing scope or the monitor for illuminating the patient's airway anatomy, for example, the patient's vocal cords, trachea, etc. In an embodiment, the intubation apparatus comprises an energy storage device for powering the light source.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific components and methods disclosed herein.

FIG. 1 exemplarily illustrates a side orthogonal view of an intubation apparatus.

FIG. 2A exemplarily illustrates a side orthogonal view of a guiding stylet coaxially enclosed by a tubular sheath and an endotracheal tube, and controlled by a control lever.

FIG. 2B exemplarily illustrates a sectional view of the guiding stylet coaxially enclosed by a tubular sheath and the endotracheal tube taken along a section A-A′ shown in FIG. 2A.

FIGS. 3A-3B exemplarily illustrate side orthogonal views of the intubation apparatus, showing embodiments of a receiving member that receives and detachably attaches the guiding stylet to an elongate support member of the intubation apparatus.

FIG. 4A exemplarily illustrates the guiding stylet coaxially enclosed by a tubular sheath that is advanced an extended distance over the adjustable distal tip of the guiding stylet.

FIG. 4B exemplarily illustrates bending of the adjustable distal tip at a distal end of the guiding stylet.

FIG. 5 exemplarily illustrates an embodiment of the intubation apparatus, showing the guiding stylet coaxially enclosed by the tubular sheath and the endotracheal tube and inserted into an opening defined on the elongate support member.

FIG. 6A exemplarily illustrates a partially orthogonal view of an embodiment of the intubation apparatus, showing the guiding stylet coaxially enclosed by the tubular sheath, inserted into one of the openings defined on an outer surface of the elongate support member, and contained within one of the channels defined within the elongate support member.

FIG. 6B exemplarily illustrates an enlarged view of a distal end of a blade element of the intubation apparatus shown in FIG. 6A, showing a viewing lens and a light port.

FIG. 7A exemplarily illustrates a partial view of the intubation apparatus, showing channels extending into the blade element.

FIGS. 7B-7C exemplarily illustrate enlarged views showing the channels extending into the blade element.

FIG. 8A exemplarily illustrates a partial view of the intubation apparatus, showing the channels extending into the blade element.

FIG. 8B exemplarily illustrates a sectional view of the blade element taken along a section B-B′ shown in FIG. 8A, showing a viewing lens and a light port housed on the distal end of the blade element.

FIG. 8C exemplarily illustrates a sectional view of the blade element taken along a section B-B′ shown in FIG. 8A, showing a viewing lens channel and a light channel defined within the blade element.

FIG. 9A exemplarily illustrates a viewing scope operably coupled to a proximal section of the elongate support member.

FIG. 9B exemplarily illustrates a monitor and a light source operably coupled to the proximal section of the elongate support member.

FIGS. 10A-10C exemplarily illustrate advancement of the tubular sheath over the adjustable distal tip of the guiding stylet for guiding an endotracheal tube into a trachea of a patient during intubation using the intubation apparatus.

FIG. 11 illustrates a method for performing controlled intubation at a target area in a trachea of a patient with minimal movement and effort and with greater precision.

### DETAILED DESCRIPTION OF THE INVENTION

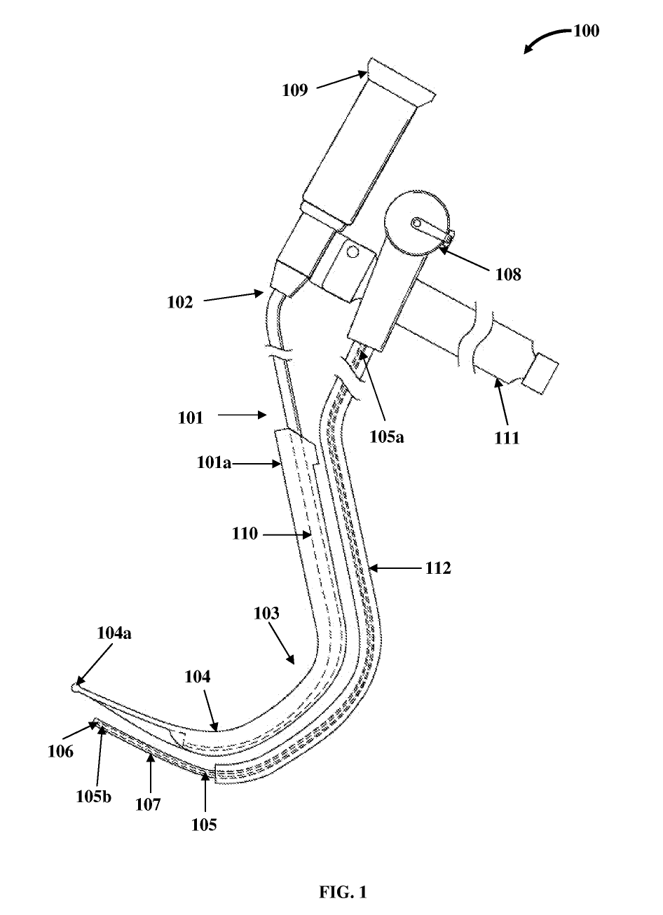

FIG. 1 exemplarily illustrates a side orthogonal view of an intubation apparatus 100. The intubation apparatus 100 disclosed herein comprises an elongate support member 101, a blade element 104, a guiding stylet 105, and a tubular sheath 107 that slidably and coaxially encloses the guiding stylet 105. The tubular sheath 107 coaxially surrounds the guiding stylet 105. The elongate support member 101 comprises a proximal section 102 and a distal section 103. The proximal section 102 of the elongate support member 101 is, for example, a generally cylindrical section that can be held and gripped by an operator of the intubation apparatus 100. The distal section 103 of the elongate support member 101 curvedly extends from the proximal section 102 of the elongate support member 101. The curvature of the distal section 103 of the elongate support member 101 is configured to conform to the curvature of a human oral and pharyngeal passageway. The elongate support member 101 is made of, for example, a metal.

A laryngoscope handle 111 made of, for example, a metal, is coupled to the proximal section 102 of the elongate support member 101. The laryngoscope handle 111 enables an operator of the intubation apparatus 100 to grasp the elongate support member 101 during an intubation procedure. Intubation refers to insertion of a flexible tube, for example, into a trachea (not shown) of a patient to maintain an open airway or to serve as a conduit for administering, for example, medication, drugs, etc. The intubation apparatus 100 disclosed herein can be manufactured in different sizes, for example, a small size, a large size, a pediatric size, an infant size, etc.

The blade element 104 of the intubation apparatus 100 is configured on the curvedly extended distal section 103 of the elongate support member 101 for maneuvering the intubation apparatus 100 into an oral cavity of a patient. In an embodiment, the blade element 104 forms the distal section 103 of the elongate support member 101. The blade element 104 has a curvature of, for example, about 20 degrees to about 40 degrees.

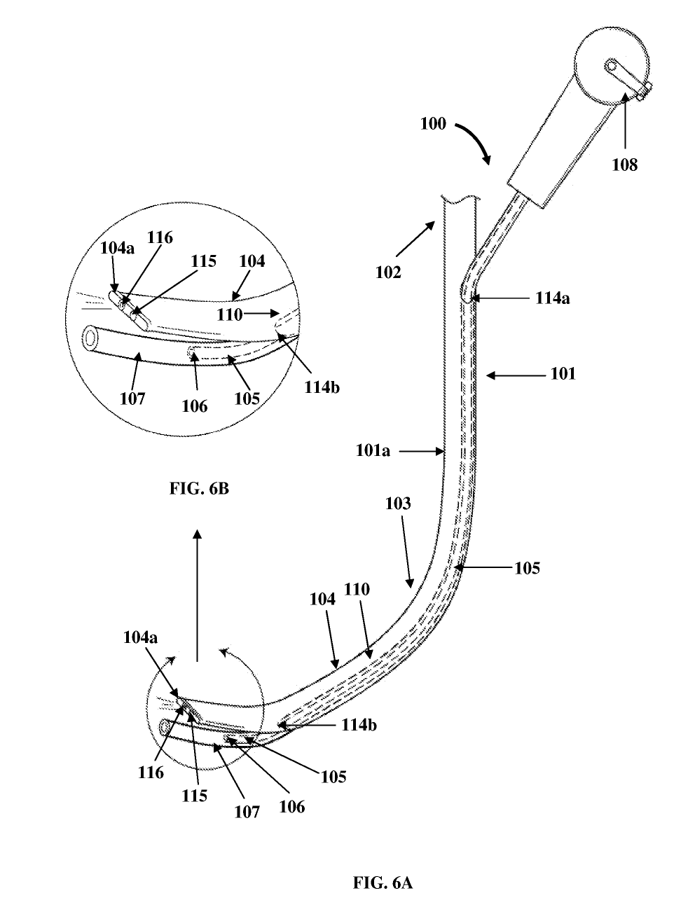

The guiding stylet 105 of the intubation apparatus 100 is detachably attached to the elongate support member 101. The guiding stylet 105 substantially follows the curvature of the elongate support member 101 and the blade element 104 for guiding an endotracheal tube 112 slidably and coaxially disposed over the tubular sheath 107 into the patient's trachea. The endotracheal tube 112 is slidably and coaxially mounted on the exterior of the guiding stylet 105. The guiding stylet 105 follows the curvature of the elongate support member 101 and the blade element 104, which allows the guiding stylet 105 to be positioned and to point to a target area where a viewing lens 115 housed on a distal end 104a of the blade element 104 points, as exemplarily illustrated in FIGS. 6A-6B. The distal end 104a of the blade element 104 is curved slightly upwards and houses the viewing lens 115 as exemplarily illustrated in FIG. 6B.

The guiding stylet 105 and the coaxial endotracheal tube 112 of the intubation apparatus 100 can be configured with different lengths. In an example, the endotracheal tube 112 is a double lumen endotracheal tube 112. The guiding stylet 105 can be of different lengths for different endotracheal tubes 112. The guiding stylet 105 comprises an adjustable distal tip 106 at a distal end 105b of the guiding stylet 105. The guiding stylet 105 is constructed from a non-traumatic material, for example, molded plastic, a soft metal, a stiff non-metallic material, etc. The guiding stylet 105 is used to provide rigidity and maintain patency of the intubation apparatus 100. In an embodiment, the endotracheal tube 112 is slid over the tubular sheath 107 from the bottom, that is, via the adjustable distal tip 106 of the guiding stylet 105. In another embodiment, the endotracheal tube 112 is slid over the tubular sheath 107 from a proximal end 105a of the guiding stylet 105.

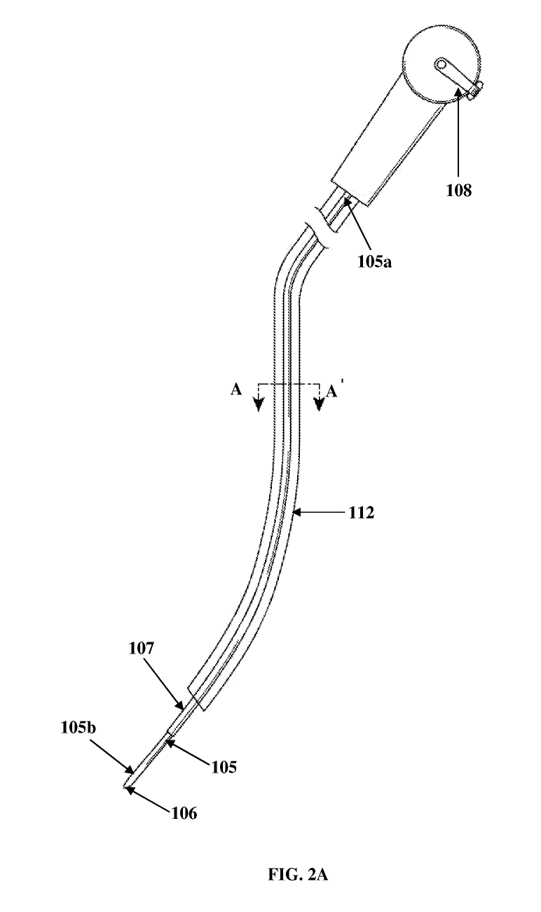

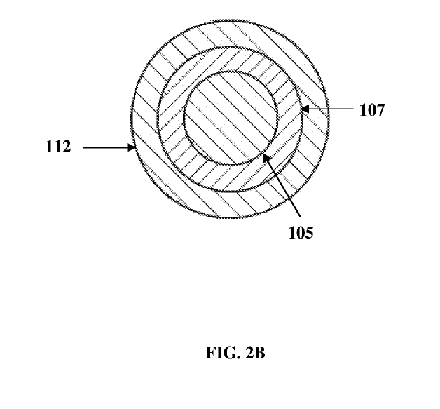

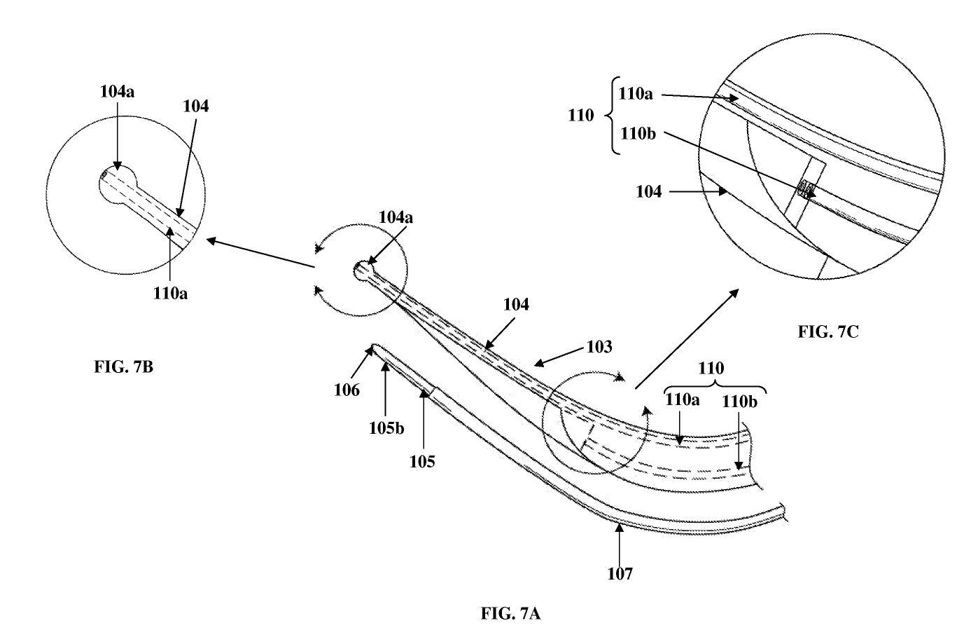

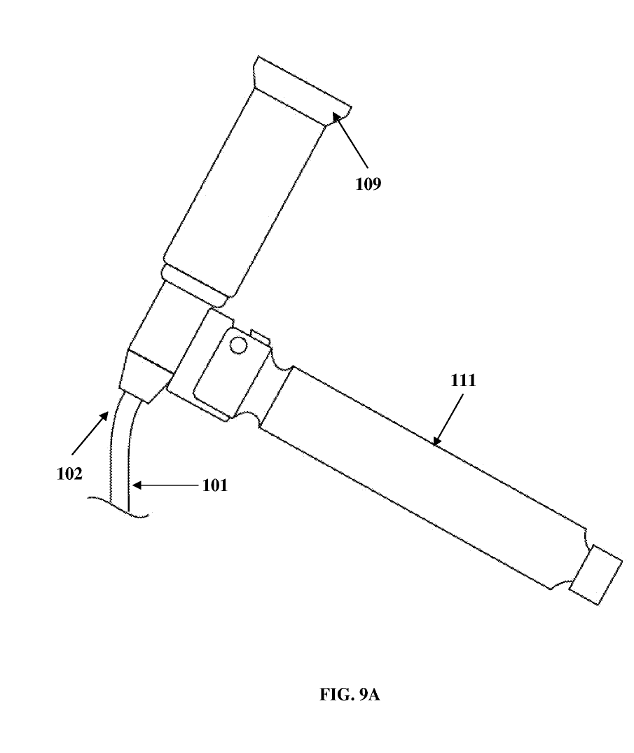

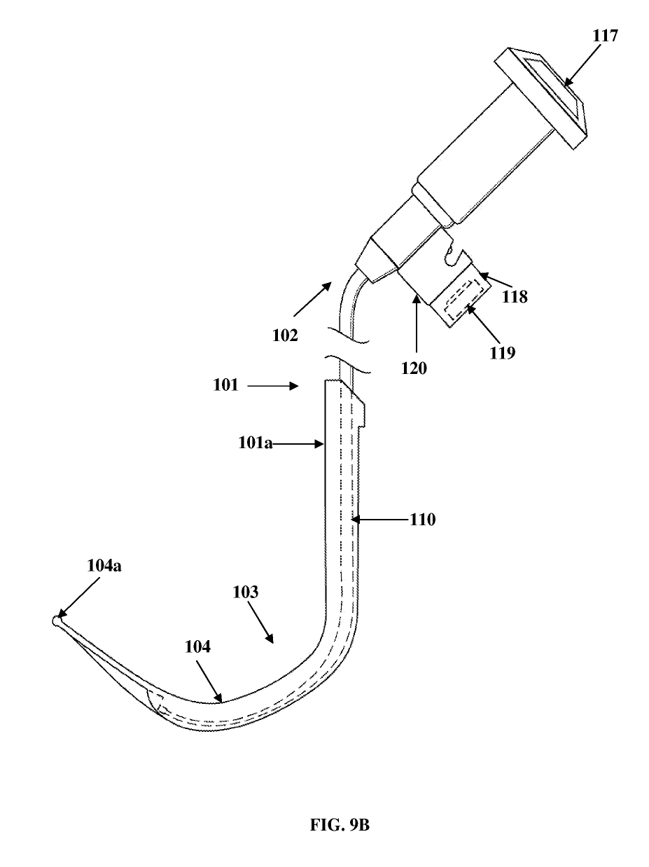

The tubular sheath 107 of the intubation apparatus 100 slidably and coaxially encloses the guiding stylet 105 as disclosed in the detailed description of FIGS. 2A-2B. The tubular sheath 107 extends from the proximal end 105a of the guiding stylet 105 to the distal end 105b of the guiding stylet 105, a few centimeters from the adjustable distal tip 106 of the guiding stylet 105. The intubation apparatus 100 further comprises a control lever 108 extending outwardly from the proximal end 105a of the guiding stylet 105 for controlling movement and orientation of the adjustable distal tip 106 of the guiding stylet 105. The intubation apparatus 100 further comprises one or more channels 110 defined within the elongate support member 101 for providing a passageway from the proximal section 102 of the elongate support member 101 to the distal section 103 of the elongate support member 101 as disclosed in the detailed description of FIGS. 6A-6B and FIGS. 7A-7C. The intubation apparatus 100 further comprises a viewing scope 109 as disclosed in the detailed description of FIG. 9A.

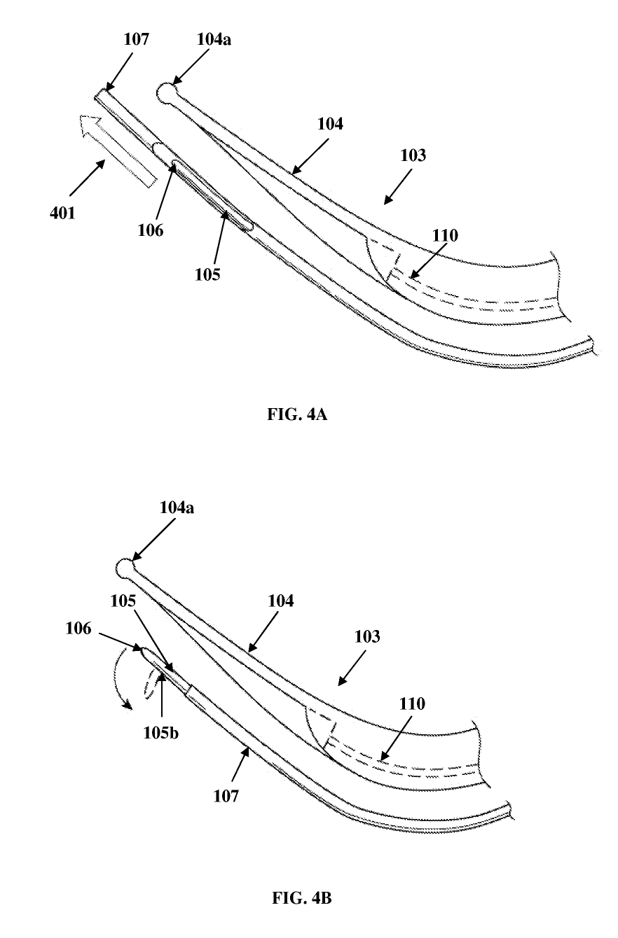

FIG. 2A exemplarily illustrates a side orthogonal view of a guiding stylet 105 coaxially enclosed by a tubular sheath 107 and an endotracheal tube 112, and controlled by a control lever 108. The tubular sheath 107 extends along a length of the guiding stylet 105. The tubular sheath 107 is constructed from, for example, a molded plastic, a soft metal, etc. The tubular sheath 107 extends beyond the blade element 104 and advances an extended distance 401 into the trachea as exemplarily illustrated in FIG. 4A. The endotracheal tube 112 is slidably and coaxially guided over the advanced tubular sheath 107 into the patient's trachea.

The control lever 108 extends outwardly from a proximal end 105a of the guiding stylet 105 for controlling movement and orientation of the adjustable distal tip 106 of the guiding stylet 105. The control lever 108 is, for example, a small wheel, a small lever, etc. The control lever 108 comprises an internal assembly (not shown) positioned within the guiding stylet 105 that connects the control lever 108 at the proximal end 105a of the guiding stylet 105 to the adjustable distal tip 106 at the distal end 105b of the guiding stylet 105. In an example, the internal assembly is a wire positioned within the guiding stylet 105 that runs throughout the length of the guiding stylet 105. In an embodiment, the control lever 108 is of a substantially small size to allow insertion of the endotracheal tube 112 from the top of the control lever 108 and then over the guiding stylet 105 enclosed by the tubular sheath 107.

FIG. 2B exemplarily illustrates a sectional view of the guiding stylet 105 coaxially enclosed by a tubular sheath 107 and an endotracheal tube 112 taken along a section A-A′ shown in FIG. 2A. The tubular sheath 107 slidably and coaxially encloses the guiding stylet 105. The tubular sheath 107 extends along a length of the guiding stylet 105 and advances into the trachea by sliding over the length of the guiding stylet 105 as exemplarily illustrated in FIG. 2A and then over the adjustable distal tip 106 of the guiding stylet 105 as exemplarily illustrated in FIG. 4A. For example, the tubular sheath 107 can be extended and protruded several centimeters in the patient's trachea. The coaxial endotracheal tube 112 is slidably and coaxially disposed over the tubular sheath 107 and guided into the trachea.

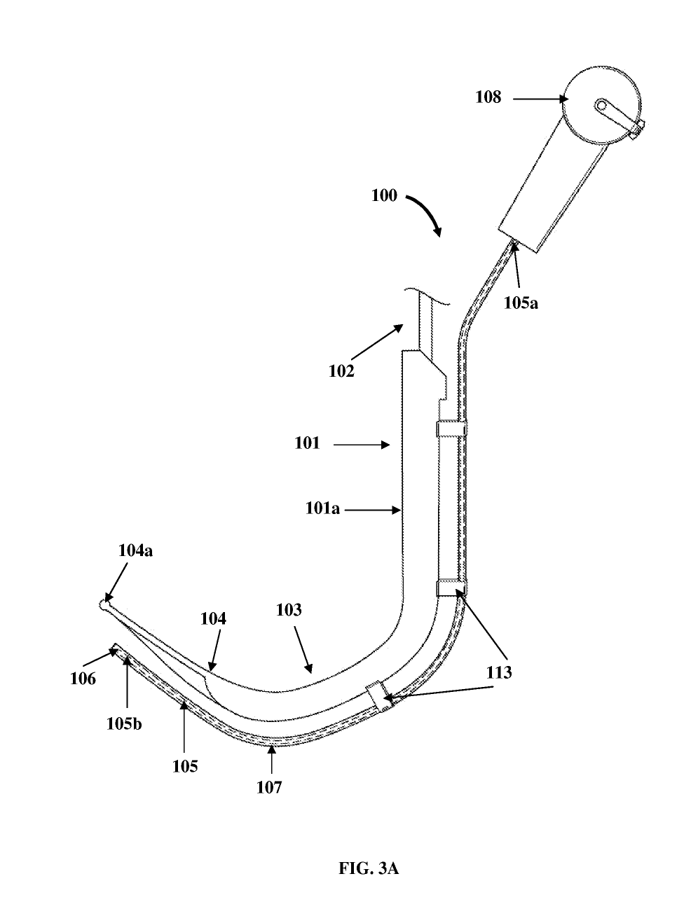

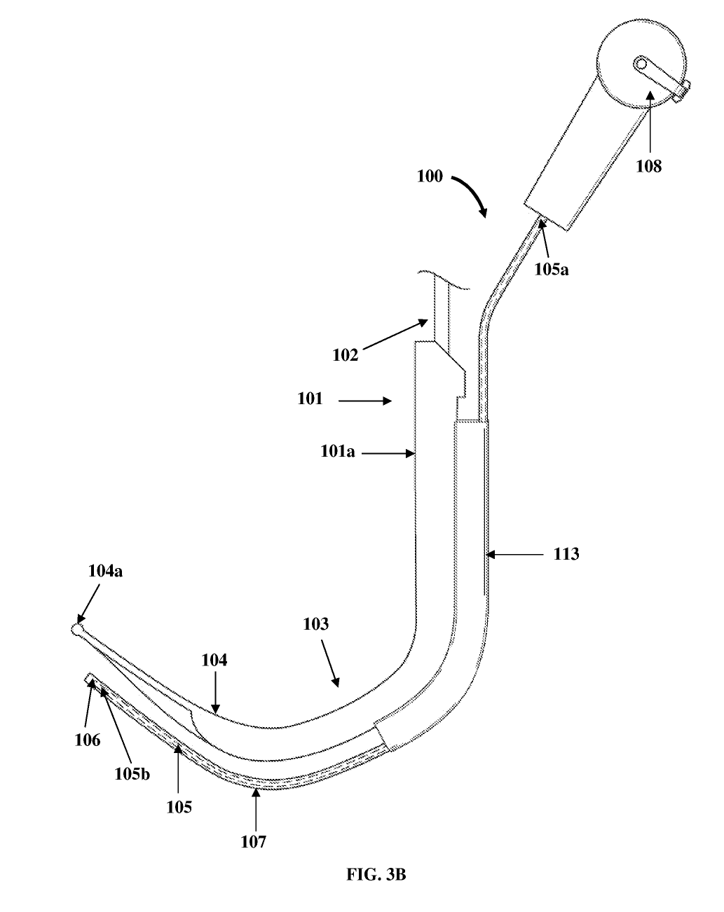

FIGS. 3A-3B exemplarily illustrate side orthogonal views of the intubation apparatus 100, showing embodiments of a receiving member 113 that receives and detachably attaches the guiding stylet 105 to the elongate support member 101 of the intubation apparatus 100. The guiding stylet 105 is inserted into the receiving member 113. The receiving member 113 is defined on an outer surface 101a of the elongate support member 101 for receiving the guiding stylet 105. The receiving member 113 further receives the tubular sheath 107 and the endotracheal tube 112 that slidably and coaxially enclose the guiding stylet 105. In an embodiment, the receiving member 113 is configured as multiple fasteners, for example, plastic ring type fasteners, that receive the guiding stylet 105 enclosed by the tubular sheath 107 as exemplarily illustrated in FIG. 3A. In this embodiment, when the guiding stylet 105, the tubular sheath 107, and the endotracheal tube 112 are positioned for intubation, the fasteners 113 are tightened for fastening the enclosed guiding stylet 105 to the elongate support member 101. In another embodiment, the receiving member 113 is configured as a sleeve defined on the outer surface 101a of the elongate support member 101 for receiving the guiding stylet 105 enclosed by the tubular sheath 107 as exemplarily illustrated in FIG. 3B. The endotracheal tube 112 is slidably and coaxially guided over the tubular sheath 107 that encloses the guiding stylet 105 within the sleeve configured receiving member 113.

FIG. 4A exemplarily illustrates the guiding stylet 105 coaxially enclosed by a tubular sheath 107 that is advanced an extended distance 401 over the adjustable distal tip 106 of the guiding stylet 105. The tubular sheath 107 extends along the length of the guiding stylet 105 and advances an extended distance 401 into the trachea by sliding over the length of the guiding stylet 105 and then over the adjustable distal tip 106 of the guiding stylet 105. The tubular sheath 107 extends over the guiding stylet 105 and advances into the trachea. The tubular sheath 107 thus provides extendibility to the intubation apparatus 100 disclosed herein. When the tubular sheath 107 is advanced past the vocal cords into the trachea, the endotracheal tube 112 is slid over the tubular sheath 107 and introduced into the trachea. The tubular sheath 107 can move beyond the blade element 104 of the intubation apparatus 100. For example, the tubular sheath 107 is extendable beyond the blade element 104 from about 3 centimeters to about 5 centimeters into the trachea.

In an embodiment, the movement of the tubular sheath 107 is controlled manually and is advanced into the patient's trachea by first holding the tubular sheath 107 in an operator's free hand, and then sliding the tubular sheath 107 over the length of the guiding stylet 105 and over the adjustable distal tip 106 of the guiding stylet 105 into the trachea. The additional extendibility provided by the tubular sheath 107 allows the operator of the intubation apparatus 100 to complete the intubation process without having to repeatedly move or adjust either the guiding stylet 105 or the elongate support member 101 in order to obtain a better visualization of the patient's airway anatomy, for example, the patient's vocal cords, trachea, etc.

FIG. 4B exemplarily illustrates bending of the adjustable distal tip 106 at a distal end 105b of the guiding stylet 105. The adjustable distal tip 106 bends in an upward direction and in a downward direction. For example, the adjustable distal tip 106 of the guiding stylet 105 bends from about 120 degrees in an upward direction to about −30 degrees in a downward direction. In another example, the adjustable distal tip 106 of the guiding stylet 105 can move 90 degrees in an upward direction or 90 degrees in a downward direction. The control lever 108, exemplarily illustrated in FIG. 1, FIG. 2A, and FIGS. 3A-3B, controls the movement and the orientation of the adjustable distal tip 106 of the guiding stylet 105.

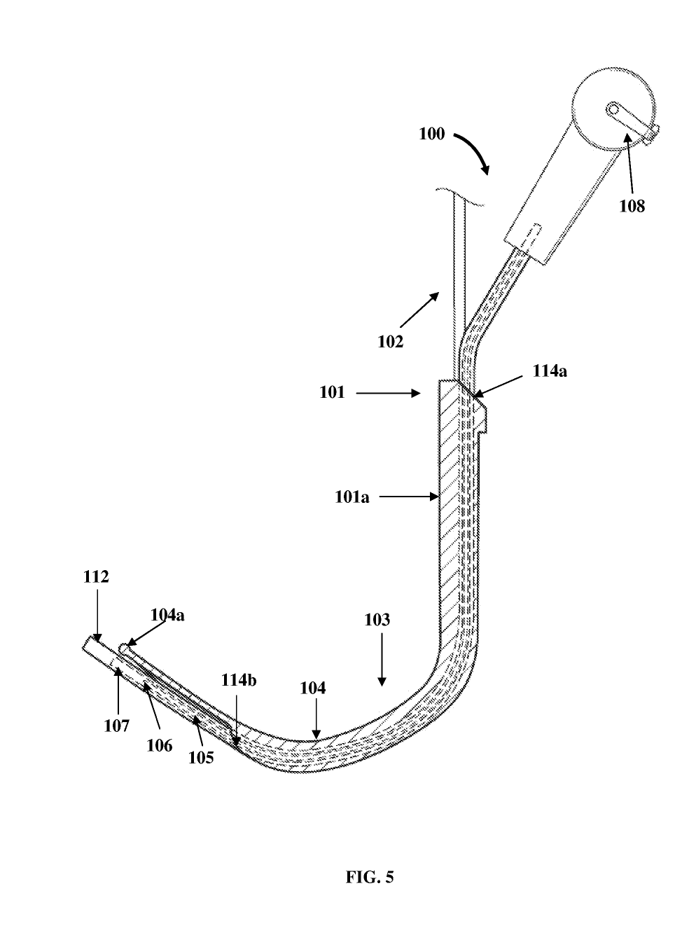

FIG. 5 exemplarily illustrates an embodiment of the intubation apparatus 100, showing the guiding stylet 105 coaxially enclosed by the tubular sheath 107 and the endotracheal tube 112 and inserted into an opening 114a defined on the elongate support member 101. In an embodiment, the intubation apparatus 100 further comprises one or more openings 114a and 114b defined on the outer surface 101a of the elongate support member 101 for inserting the guiding stylet 105, the tubular sheath 107, and the endotracheal tube 112. An operator of the intubation apparatus 100 can insert the guiding stylet 105 through the opening 114a and advance the guiding stylet 105 such that the adjustable distal tip 106 of the guiding stylet 105 comes out through the other opening 114b. The operator can then insert the tubular sheath 107 through the opening 114a over the length of the guiding stylet 105 and slide the tubular sheath 107 to come out through the other opening 114b over the adjustable distal tip 106 of the guiding stylet 105. The tubular sheath 107 is advanced an extended distance 401 over the adjustable distal tip 106 of the guiding stylet 105 as exemplarily illustrated in FIG. 4A. The operator can then insert the endotracheal tube 112 through the opening 114a over the tubular sheath 107 and slide the endotracheal tube 112 to come out through the other opening 114b over the tubular sheath 107. The guiding stylet 105, the tubular sheath 107, and the endotracheal tube 112 can be inserted into the elongate support member 101 through one of the openings 114a and 114b. The guiding stylet 105, the tubular sheath 107, and the endotracheal tube 112 follow the curvature of the blade element 104 beyond the distal end 104a of the blade element 104.

FIG. 6A exemplarily illustrates a partially orthogonal view of an embodiment of the intubation apparatus 100, showing the guiding stylet 105 coaxially enclosed by the tubular sheath 107, inserted into one of the openings 114a and 114b defined on the outer surface 101a of the elongate support member 101, and contained within one of the channels 110 defined within the elongate support member 101.

FIG. 6B exemplarily illustrates an enlarged view of the distal end 104a of the blade element 104 of the intubation apparatus 100 shown in FIG. 6A, showing a viewing lens 115 and a light port 116. The viewing lens 115 is housed on the distal end 104a of the blade element 104 for viewing the patient's vocal chords and the trachea. The viewing lens 115 collects optical images for transmission through the channels 110 defined within the elongate support member 101. The viewing lens 115 is, for example, a fiber optic lens, an infrared imaging lens, an ultrasound imaging lens, a wide angle lens, etc. The visualization provided by the fiber optic lens in the presence of secretions, blood or severe inflammatory reactions may sometimes be distorted or blocked. The infrared imaging lens or the ultrasound imaging lens provides unobtrusive viewing of the patient's trachea, as these lenses do not distort or block viewing in the presence of secretions, blood or severe inflammatory reactions. The infrared imaging lens or the ultrasound imaging lens provide clear visualizations even in the presence of secretions, blood or severe inflammatory reactions. The wide angle lens provides a panoramic view of the patient's airway anatomy. The light port 116 is also housed on the distal end 104a of the blade element 104. The light port 116 is, for example, an opening at the end of a light channel 110a exemplarily illustrated in FIGS. 7A-7C, through which light passing through the light channel 110a exits. The light port 116 thus illuminates the field to be visualized. The light is provided, for example, by a light source 118 positioned on the proximal section 102 of the elongate support member 101 as exemplarily illustrated in FIG. 9B.

FIG. 7A exemplarily illustrates a partial view of the intubation apparatus 100, showing channels 110 extending into the blade element 104. The channels 110 extend from the proximal section 102 of the elongate support member 101 to the distal end 104a of the blade element 104. The channels 110 are, for example, a bundle of optic fibers. The channels 110 defined within the elongate support member 101 provide a passageway from the proximal section 102 of the elongate support member 101 to the distal section 103 of the elongate support member 101.

In an embodiment, one of the channels 110 is a light channel 110a defined within the elongate support member 101, which extends into the blade element 104 configured on the distal section 103 of the elongate support member 101. The light channel 110a extends from the proximal section 102 of the elongate support member 101 to the distal end 104a of the blade element 104. The light channel 110a transmits light from a light source 118 positioned on the proximal section 102 of the elongate support member 101, as exemplarily illustrated in FIG. 9B, to a light port 116 housed on the distal end 104a of the blade element 104. In this embodiment, the light channel 110a communicates with the light source 118 of a sufficiently high intensity to illuminate the field. For example, the light source 118 is a halogen bulb powered by an energy storage device 119 as exemplarily illustrated in FIG. 9B.

In another embodiment, one of the channels 110 is a viewing lens channel 110b defined within the elongate support member 101 for guiding light from a viewing lens 115 housed on the distal end 104a of the blade element 104 to a viewing scope 109 or a monitor 117 operably coupled to the proximal section 102 of the elongate support member 101 as exemplarily illustrated in FIGS. 9A-9B.

FIGS. 7B-7C exemplarily illustrate enlarged views showing the channels 110 extending into the blade element 104. As exemplarily illustrated in FIG. 7B, the channel 110a terminates at the distal end 104a of the blade element 104. The channel 110a is, for example, the light channel 110a disclosed in the detailed description of FIG. 7A. In an embodiment, one of the channels 110 as exemplarily illustrated in FIG. 7C, may be used for containing the guiding stylet 105 slidably and coaxially enclosed by the tubular sheath 107, while another one of the channels 110 may be the viewing lens channel 110b.

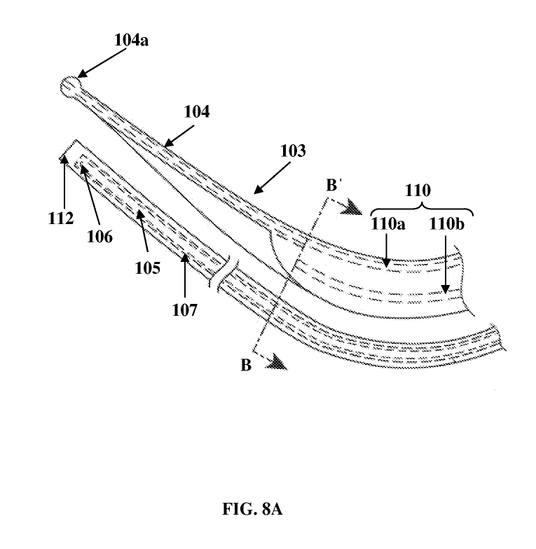

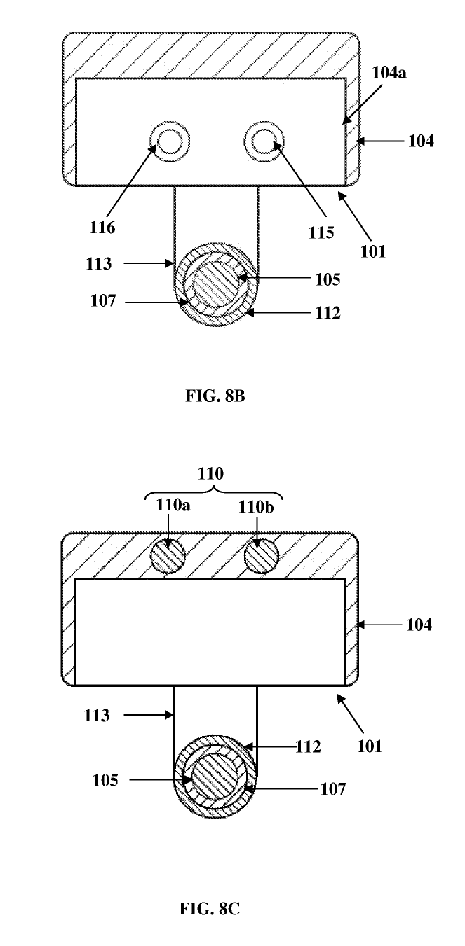

FIG. 8A exemplarily illustrates a partial view of the intubation apparatus 100, showing the channels 110 extending into the blade element 104. FIG. 8A shows positioning of the endotracheal tube 112 coaxially disposed over the tubular sheath 107 that coaxially encloses the guiding stylet 105 relative to the blade element 104.

FIG. 8B exemplarily illustrates a sectional view of the blade element 104 taken along a section B-B′ shown in FIG. 8A, showing a viewing lens 115 and a light port 116 housed on the distal end 104a of the blade element 104. The viewing lens 115 and the light port 116 are disclosed in the detailed description of FIG. 6B. FIG. 8B also illustrates the receiving member 113 that receives and attaches the guiding stylet 105 coaxially enclosed by the tubular sheath 107 and the endotracheal tube 112 to the elongate support member 101.

FIG. 8C exemplarily illustrates a sectional view of the blade element 104 taken along a section B-B′ shown in FIG. 8A, showing a viewing lens channel 110b and a light channel 110a defined within the blade element 104. The viewing lens channel 110b and the light channel 110a are disclosed in the detailed description of FIG. 7A. FIGS. 8B-8C also illustrate the receiving member 113 that receives and attaches the guiding stylet 105 coaxially enclosed by the tubular sheath 107 and the endotracheal tube 112 to the elongate support member 101.

FIG. 9A exemplarily illustrates a viewing scope 109 operably coupled to the proximal section 102 of the elongate support member 101. The viewing scope 109 is angled towards the operator of the intubation apparatus 100. The viewing scope 109 enables viewing of the patient's vocal cords and trachea, via the viewing lens 115 housed on the distal end 104a of the blade element 104 exemplarily illustrated in FIGS. 6A-6B. The images captured by the viewing lens 115 are transmitted through the viewing lens channel 110b exemplarily illustrated in FIG. 7A, FIG. 7C, FIG. 8A, and FIG. 8C and are made available at the viewing scope 109. The operator of the intubation apparatus 100 can view remote parts of the patient's anatomy by looking into the viewing scope 109 of the intubation apparatus 100. In an embodiment, a laryngoscope handle 111 coupled to the proximal section 102 of the elongate support member 101 is configured as an alternative light source for illuminating the patient's airway anatomy, for example, the vocal cords, the trachea, etc. The laryngoscope handle 111 can also be used as a handle to grasp the intubation apparatus 100. The laryngoscope handle 111 configured as the light source can be positioned, for example, on the proximal section 102 of the elongate support member 101 and below the viewing scope 109 for illuminating the patient's airway anatomy. When the laryngoscope handle 111 is not used, an external light source 118, for example, a rechargeable battery as exemplarily illustrated in FIG. 9B is used.

FIG. 9B exemplarily illustrates a monitor 117 and a light source 118 operably coupled to the proximal section 102 of the elongate support member 101. The monitor 117 is, for example, a liquid crystal display (LCD) monitor, a light emitting diode (LED) monitor, etc. The monitor 117 enables viewing of the patient's vocal cords and trachea via the viewing lens 115, exemplarily illustrated in FIGS. 6A-6B, housed on the distal end 104a of the blade element 104. In an embodiment, the monitor 117 is configured for enabling viewing of the patient's vocal cords and trachea via an ultrasound imaging lens housed on the distal end 104a of the blade element 104. In an embodiment, an image capture device, for example, a camera (not shown) is housed on the distal end 104a of the blade element 104, in place of the viewing lens 115. The image capture device is operably coupled to the distal end 104a of the blade element 104 configured on the distal section 103 of the elongate support member 101. The image capture device is configured to transmit optical images of a target area to the monitor 117 or a video recording apparatus such as a video cassette recorder (not shown) operably coupled to the proximal section 102 of the elongate support member 101 via one of the channels 110, for example, 110b exemplarily illustrated in FIG. 7A, FIG. 7C, FIG. 8A, and FIG. 8C, defined within the elongate support member 101.

As exemplarily illustrated in FIG. 9B, a light source 118 is positioned, for example, on the proximal section 102 of the elongate support member 101 and below the monitor 117 for illuminating the patient's airway anatomy, for example, the patient's vocal cords, trachea, etc. The light source 118 is connected to the proximal section 102 of the elongate support member 101, for example, by an adaptor element 120 positioned immediately below the monitor 117. In an embodiment, a battery operated light source 118 or an external light source 118 is used for illuminating the patient's airway anatomy. In another embodiment, the light source 118 is provided by a combination of both a laryngoscope and a rechargeable battery, for example, a lithium battery. A regular laryngoscope can be attached to the proximal section 102 of the elongate support member 101 to provide the light source 118. In an embodiment, an energy storage device 119, for example, a lithium battery is provided for powering the light source 118 that illuminates the patient's airway anatomy.

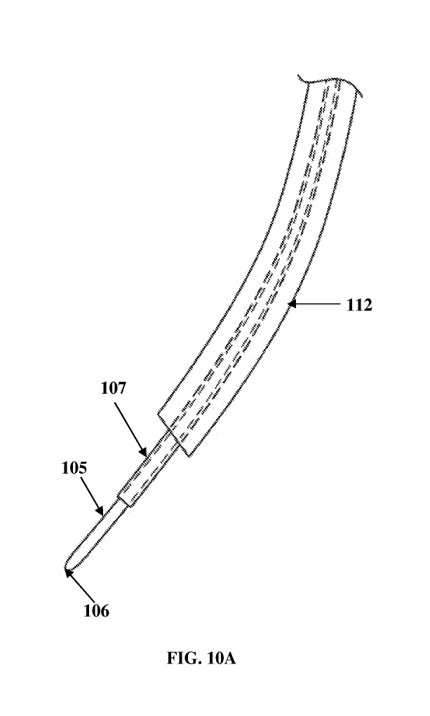

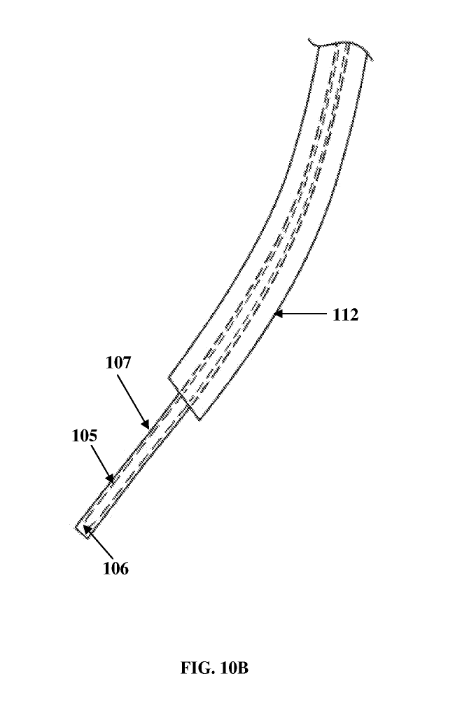

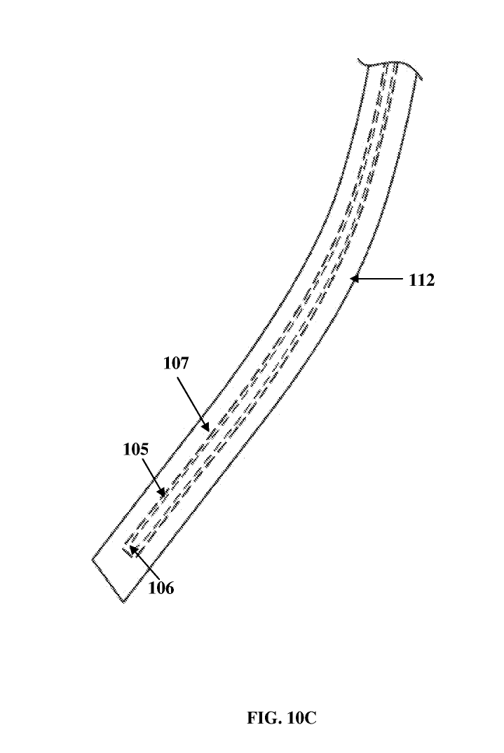

FIGS. 10A-10C exemplarily illustrate advancement of the tubular sheath 107 over the adjustable distal tip 106 of the guiding stylet 105 for guiding an endotracheal tube 112 into the trachea of a patient during intubation using the intubation apparatus 100. The intubation apparatus 100 is exemplarily illustrated in FIG. 1. FIG. 10A exemplarily illustrates the endotracheal tube 112 slidably and coaxially disposed over the tubular sheath 107 which slidably and coaxially encloses the guiding stylet 105. The guiding stylet 105 can be moved forward or manipulated into a target area in the patient's airway anatomy. FIG. 10B exemplarily illustrates the tubular sheath 107 extended along a length of the guiding stylet 105 and advanced an extended distance 401 into the trachea (not shown) as exemplarily illustrated in FIG. 4A. The tubular sheath 107 is advanced an extended distance 401 by sliding the tubular sheath 107 over the length of the guiding stylet 105 and then over the adjustable distal tip 106 of the guiding stylet 105. The tubular sheath 107 is advanced the extended distance 401 into the trachea after viewing the patient's vocal cords via the viewing lens 115 housed on the distal end 104a of the blade element 104. FIG. 10C exemplarily illustrates the endotracheal tube 112 slidably and coaxially enclosing the advanced tubular sheath 107.

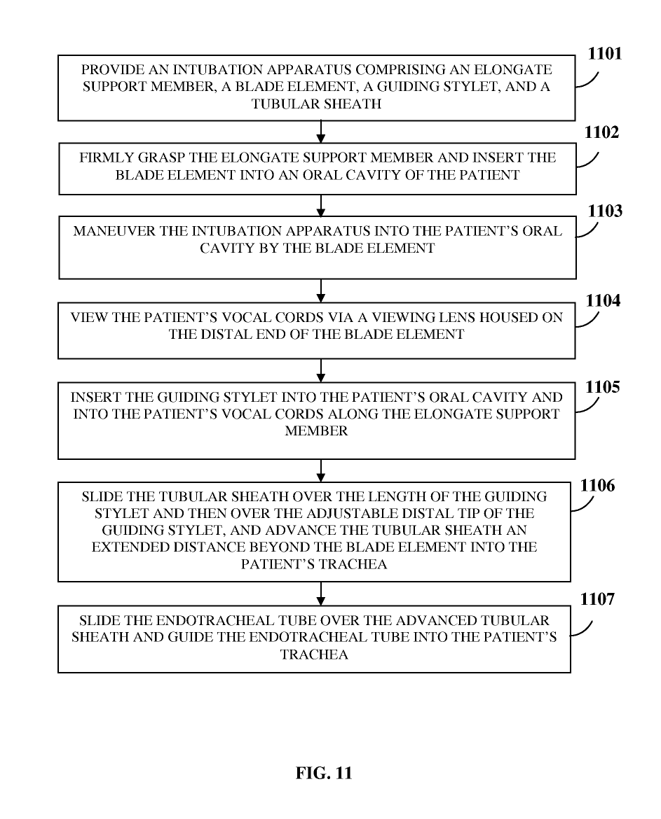

FIG. 11 illustrates a method for performing controlled intubation at a target area in a trachea of a patient with minimal movement and effort and with greater precision. An intubation apparatus 100 comprising an elongate support member 101, a blade element 104, a guiding stylet 105, and a tubular sheath 107 as disclosed in the detailed description of FIGS. 1-9B is provided 1101. An operator of the intubation apparatus 100 firmly grasps the elongate support member 101 of the intubation apparatus 100 and inserts 1102 the blade element 104 of the intubation apparatus 100 into the oral cavity of the patient. The operator uses the blade element 104 to maneuver 1103 the intubation apparatus 100 into the patient's oral cavity. The operator illuminates the patient's airway anatomy using the light source 118 positioned on the proximal section 102 of the elongate support member 101 exemplarily illustrated in FIG. 9B. The operator views 1104 the patient's vocal cords via the viewing lens 115 housed on the distal end 104a of the blade element 104. The operator inserts 1105 the guiding stylet 105 of the intubation apparatus 100 into the patient's oral cavity and into the patient's vocal cords along the elongate support member 101, where the guiding stylet 105 substantially follows the curvature of the elongate support member 101 and the blade element 104. The operator may control movement and orientation of the adjustable distal tip 106 of the guiding stylet 105 using the control lever 108 extending outwardly from the proximal end 105a of the guiding stylet 105 to obtain a better view of the patient's vocal cords. The operator slides 1106 the tubular sheath 107 of the intubation apparatus 100 over the length of the guiding stylet 105 and then over the adjustable distal tip 106 of the guiding stylet 105 and advances the tubular sheath 107 an extended distance 401 beyond the blade element 104 into the patient's trachea (not shown) as exemplarily illustrated in FIG. 4A. The operator then slides 1107 the endotracheal tube 112 over the advanced tubular sheath 107 and guides the endotracheal tube 112 into the patient's trachea. The advancement of the tubular sheath 107 over the adjustable distal tip 106 of the guiding stylet 105 to the extended distance 401 controllably guides the endotracheal tube 112 to the target area in the patient's trachea for performing the controlled intubation.

Consider an example where an operator performs controlled intubation. The operator grasps the laryngoscope handle 111 coupled to the proximal section 102 of the elongate support member 101 of the intubation apparatus 100 exemplarily illustrated in FIG. 1 and FIG. 9A. The operator then inserts the blade element 104 configured on the distal section 103 of the elongate support member 101 into the patient's mouth. The operator advances the blade element 104 into the patient's oral cavity. The operator pushes the blade element 104 further and passes the patient's tongue such that the back of the blade element 104 rests against the patient's tongue. The viewing lens 115 housed on the distal end 104a of the blade element 104, as exemplarily illustrated in FIGS. 6A-6B, now provides an anterior view of the patient's oral cavity to the operator. The operator views the visualizations obtained by the viewing lens 115 through the viewing scope 109 operably coupled to the proximal section 102 of the elongate support member 101 exemplarily illustrated in FIG. 1 and FIG. 9A. The operator performs an upward traction on the mandible using the laryngoscope handle 111 to bring the patient's vocal cords into view. The operator further advances and manipulates the blade element 104 to bring the vocal cords into view. The blade element 104 having a curvature of about 20 degrees to about 40 degrees allows easier visualization of the vocal cords, especially in patients with a difficult anatomy.

The operator disposes the endotracheal tube 112 over the tubular sheath 107. When the vocal cords are visualized, the operator advances the guiding stylet 105 from the patient's oral cavity to the vocal cords. The operator maneuvers the adjustable distal tip 106 of the guiding stylet 105 in an upward direction or a downward direction, as exemplarily illustrated in FIG. 4B, using the control lever 108, hence bringing the vocal cords into view. On obtaining optimal visualization, the operator slides the tubular sheath 107 over the guiding stylet 105 and advances the tubular sheath 107 into the trachea. When the viewing lens 115 housed on the blade element 104 identifies the vocal cords, the operator has only to slide the tubular sheath 107 over the adjustable distal tip 106 of the guiding stylet 105 and into trachea. Without this, the guiding stylet 105 would simply move in any direction in the oral cavity and possibly away from the target area. The operator then advances the endotracheal tube 112 that is already positioned on the tubular sheath 107 into the trachea.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may affect numerous modifications thereto and changes may be made without departing from the scope and spirit of the invention in its aspects.

I claim:

1. An intubation apparatus, comprising:
an elongate support member comprising a proximal section and a distal section, wherein said distal section curvedly extends from said proximal section;
a blade element configured on said curvedly extended distal section of said elongate support member for maneuvering said intubation apparatus into an oral cavity of a patient;
a guiding stylet detachably attached to said elongate support member, said guiding stylet substantially following curvature of said elongate support member and said blade element for guiding an endotracheal tube into a trachea of said patient, wherein said guiding stylet comprises an adjustable distal tip at a distal end of said guiding stylet; and
a tubular sheath slidably and coaxially enclosing said guiding stylet, wherein said tubular sheath extends along a length of said guiding stylet and advances an extended distance into said trachea by sliding over said length of said guiding stylet and then over said adjustable distal tip of said guiding stylet, and wherein said endotracheal tube is slidably and coaxially guided over said advanced tubular sheath into said trachea.

2. The intubation apparatus of claim 1, wherein said tubular sheath extends beyond said blade element and advances said extended distance into said trachea.

3. The intubation apparatus of claim 1, further comprising a control lever extending outwardly from a proximal end of said guiding stylet for controlling movement and orientation of said adjustable distal tip of said guiding stylet.

4. The intubation apparatus of claim 1, further comprising a receiving member defined on an outer surface of said elongate support member for receiving said guiding stylet, wherein said receiving member further receives said tubular sheath and said endotracheal tube that slidably and coaxially enclose said guiding stylet.

5. The intubation apparatus of claim 1, wherein said guiding stylet slidably and coaxially enclosed by said tubular sheath is contained within one of one or more channels defined within said elongate support member.

6. The intubation apparatus of claim 1, further comprising one or more openings defined on an outer surface of said elongate support member for inserting said guiding stylet, said tubular sheath, and said endotracheal tube.

7. The intubation apparatus of claim 1, further comprising a viewing lens housed on a distal end of said blade element for viewing vocal cords and said trachea of said patient.

8. The intubation apparatus of claim 7, wherein said viewing lens is one of a fiber optic lens, an infrared imaging lens, and an ultrasound imaging lens.

9. The intubation apparatus of claim 7, further comprising a viewing scope operably coupled to said proximal section of said elongate support member for enabling viewing of vocal cords and said trachea of said patient via said viewing lens.

10. The intubation apparatus of claim 7, further comprising a monitor operably coupled to said proximal section of said elongate support member for enabling viewing of vocal cords and said trachea of said patient via said viewing lens.

11. The intubation apparatus of claim 1, further comprising one or more channels defined within said elongate support member for providing a passageway from said proximal section of said elongate support member to said distal section of said elongate support member.

12. The intubation apparatus of claim 11, wherein one of said one or more channels is a light channel defined within said elongate support member for transmitting light from a light source positioned on said proximal section of said elongate support member to a light port housed on a distal end of said blade element configured on said distal section of said elongate support member.

13. The intubation apparatus of claim 11, wherein one of said one or more channels is a viewing lens channel defined within said elongate support member for guiding light from a viewing lens housed on a distal end of said blade element configured on said distal section of said elongate support member to one of a viewing scope and a monitor operably coupled to said proximal section of said elongate support member.

14. The intubation apparatus of claim 1, wherein said adjustable distal tip of said guiding stylet bends from about 120 degrees in an upward direction to about −30 degrees in a downward direction.

15. The intubation apparatus of claim 1, wherein said blade element has a curvature of about 20 degrees to about 40 degrees.

16. The intubation apparatus of claim 1, further comprising a light source positioned on said proximal section of said elongate support member for illuminating vocal cords and said trachea of said patient.

17. The intubation apparatus of claim 16, further comprising an energy storage device for powering said light source.

18. A method for performing controlled intubation at a target area in a trachea of a patient with minimal movement and effort and with greater precision, comprising:
providing an intubation apparatus comprising:
an elongate support member comprising a proximal section and a distal section, wherein said distal section curvedly extends from said proximal section;
a blade element configured on said curvedly extended distal section of said elongate support member;
a guiding stylet detachably attached to said elongate support member, wherein said guiding stylet comprises an adjustable distal tip at a distal end of said guiding stylet; and
a tubular sheath slidably and coaxially enclosing said guiding stylet, wherein said tubular sheath extends along a length of said guiding stylet;
grasping firmly said elongate support member of said intubation apparatus, and inserting said blade element of said intubation apparatus into an oral cavity of said patient;
maneuvering said intubation apparatus into said oral cavity of said patient by said blade element;
viewing vocal cords of said patient via a viewing lens housed on a distal end of said blade element;
inserting said guiding stylet of said intubation apparatus into said oral cavity and into said vocal cords of said patient along said elongate support member, wherein said guiding stylet substantially follows curvature of said elongate support member and said blade element;
sliding said tubular sheath of said intubation apparatus over said length of said guiding stylet and then over said adjustable distal tip of said guiding stylet, and advancing said tubular sheath an extended distance beyond said blade element into said trachea of said patient; and
sliding said endotracheal tube over said advanced tubular sheath and guiding said endotracheal tube into said trachea of said patient;
whereby said advancement of said tubular sheath over said adjustable distal tip of said guiding stylet to said extended distance controllably guides said endotracheal tube to said target area in said trachea of said patient for performing said controlled intubation.

19. The method of claim 18, further comprising controlling movement and orientation of said adjustable distal tip of said guiding stylet using a control lever extending outwardly from a proximal end of said guiding stylet.

20. The method of claim 18, further comprising illuminating said vocal cords and said trachea of said patient using a light source positioned on said proximal section of said elongate support member of said intubation apparatus.

21. The method of claim 18, further comprising providing a passageway from said proximal section of said elongate support member to said distal section of said elongate support member by one or more channels defined within said elongate support member.

22. The method of claim 21, wherein one of said one or more channels is a light channel defined within said elongate support member for transmitting light from a light source positioned on said proximal section of said elongate support member to a light port housed on a distal end of said blade element configured on said distal section of said elongate support member.

23. The method of claim 21, wherein one of said one or more channels is a viewing lens channel defined within said elongate support member for guiding light from a viewing lens housed on a distal end of said blade element configured on said distal section of said elongate support member to one of a viewing scope and a monitor operably coupled to said proximal section of said elongate support member.

24. The method of claim 18, further comprising bending said adjustable distal tip of said guiding stylet from about 120 degrees in an upward direction to about −30 degrees in a downward direction using a control lever extending outwardly from a proximal end of said guiding stylet.
